(19) United States
(12) Patent Application Publication
(10) Pub. No.: US 2004/0049173 A1
(43) Pub. Date: Mar. 11, 2004

(54) Trocar shield actuator mechanism

(75) Inventors: Michael James White, Leeds (GB); Stuart Skelton Moran, Leeds (GB)

Correspondence Address: BEYER WEAVER & THOMAS LLP, P.O. BOX 778, BERKELEY, CA, 94704-0778, US

(73) Assignee: SURGICAL INNOVATIONS LTD.

(21) Appl. No.: 10/613,505

(22) Filed: Jul. 3, 2003

(30) Foreign Application Priority Data
Jul. 5, 2002 (GB) 0215537.2

(51) Int. Cl. A61B 17/00

(52) U.S. Cl. 606/1

(57) ABSTRACT

A trocar actuation mechanism comprising a mounting for a trocar spike, cylindrical sleeve having a proximal end, a distal end and a bush at the distal end, the sleeve being disposed within a housing and adapted to move relative to the housing between a distal position wherein the sleeve covers the trocar spike in use and a proximal position wherein the trocar spike is exposed, releasable locking means being adapted to lock the sleeve when the sleeve is moved into the distal position, an actuator moveable from an armed position wherein the sleeve may move within a housing to a disarmed position wherein the sleeve is prevented from movement, herein the locking means comprises an axially movable barrel engaging a proximal end of the sleeve and urged distally by a main spring, the barrel having a transverse aperture and further including a socket adjacent, a proximal end of the aperture, an actuator rod having an axis and being mounted in the housing and extending transversely through the aperture, the rod being movable axially between the first and second positions, the actuator rod having a shaft and a manual actuator at one end thereof, the rod further including an abutment facing towards the manual actuator, a locking plate axially movable relative to the barrel, the locking plate including a latch portion adapted to engage the actuation rod preventing movement of the rod from the second to the first position wherein in the first position of the actuation rod, the actuation rod engages the socket to lock and arm the sleeve, and wherein actuation of the manual actuator causes the rod to move to the second armed position.

This invention relates to an actuator mechanism for a protective shield for a surgical trocar.

Protective shields for trocar devices generally comprise a retractable sleeve surrounding the trocar point which serves to prevent or reduce the risk of damage to internal organs once the instrument has penetrated a patient's body wall. A mechanism is provided so that the shield is retracted as the point enters the patient, but extends to cover the point after it has penetrated the body wall. Various arrangements have been disclosed. These incorporate complex moving parts and are consequently difficult to manufacture and maintain. Accordingly there is a need for a trocar shield assembly which is of simple construction and which is robust and reliable in use.

According to a first aspect of the present invention a trocar actuator mechanism comprises:

a mounting for a trocar spike;

an axial shaft having a proximal end, a distal end and a bush at the distal end;

the shaft being disposed within a housing and adapted to move relative to the housing between a distal position wherein the trocar spike is protected in use and a proximal position wherein the trocar spike is exposed;

releasable locking means being adapted to lock the shaft when the shaft is moved into the distal position;

an actuator moveable from an armed position wherein the shaft may move within a housing to a disarmed position wherein the shaft is prevented from movement;

wherein the locking means comprises:

an axially movable portion on the proximal end of the shaft;

an actuator extending through a slot in said portion;

wherein the actuator engages proximal and distal ends of the slot to limit movement of the shaft;

the actuator being transversely movable between first and second positions, wherein in the first position the actuator engages the shaft to lock and arm the trocar and wherein manual actuation of the actuator causes the actuator to move to the second position wherein the trocar is disarmed.

According to a second aspect of the present invention a trocar actuation mechanism comprises a mounting for a trocar spike;

an axial shaft having a proximal end, a distal end and a bush at the distal end;

the shaft being disposed within a housing and adapted to move relative to the housing between a distal position wherein the trocar spike is protected in use and a proximal position wherein the trocar spike is exposed;

releasable locking means being adapted to lock the shaft when the shaft is moved into the distal position;

an actuator moveable from an armed position wherein the shaft may move within a housing to a disarmed position wherein the shaft is prevented from movement;

wherein the locking means comprises:

an axially movable barrel engaging a proximal end of the shaft and urged distally by a main spring,

the barrel having a transverse aperture and further including a socket adjacent, a proximal end of the aperture,

an actuator having an axis and being mounted in the housing and extending transversely through the aperture, the actuator being movable axially between first and second positions,

the actuator having a shaft and a manual actuator at one end thereof, the actuator further including an abutment facing towards the manual actuator,

a locking plate axially movable relative to the barrel, the locking plate including a latch portion adapted to engage the actuator preventing movement of the actuator from the second to the first position;

wherein in the first position of the actuator, the actuator engages the socket to lock and arm the sleeve, and

wherein actuation of the manual actuator causes the actuator to move to the second disarmed position.

The axial shaft may comprise a rod extending axially of the trocar and adapted to carry the shield or protector for the trocar spike. Alternatively, the axial shaft may comprise an axially located cylindrical tubular structure carrying the shield or protector surrounding a fired axial support rod carrying the trocar spike.

In a preferred embodiment a spring or other biassing means urges the actuation rod towards the first position, wherein the actuator button is caused to extend from the housing.

Preferably latch biassing means is adapted to urge the latch into engagement with the rod, preferably so that the latch engages a proximal surface of the rod.

The latch may include an aperture, for example, a slot extending longitudinally with respect to the housing.

In a preferred embodiment the transverse aperture comprises a longitudinally extending slot communicating between transverse faces of the barrel.

In a preferred embodiment the latch comprises a plate or other slidable member. The latch is preferably located between proximal and distal portions of the barrel, preferably between end pieces thereof, the latch being permitted to move axially, being urged in the distal direction by a spring or other resilient means. The actuator rod extends through the aperture or slot so that the resilient means causes a proximal end of the slot or aperture to bear agent or be urged towards the shaft of the actuator rod. In a first embodiment the shaft of the actuator rod may include a cam portion which serves to cooperate with the locking plate to urge the rod to the first position.

When the barrel moves proximally against the action of the main spring, the slot in the plate also moves proximally so that the edge thereof may no longer engage the actuator rod, allowing free movement of the actuator rod.

The actuator rod may be in any location between the first and second positions wherein it is engaged by the latch. In the first position the latch may engage the shaft or cam portion. In the first position the cam portion is also received and engaged with a socket of the barrel. The socket preferably comprises a recess, for example, a cylindrical recess surrounding the proximal end of the transverse aperture of the barrel.

Pressure on the manual actuator or button moves the actuator rod against the force of the spring to the second position, until the plate is able to move behind the abutment surface preventing movement of the rod by the spring into the first position. In a first embodiment the latch plate is caused to pass against the force of the spring to the second position, until the plate is able to move behind the abutment surface preventing movement of the rod by the spring into the first position. The barrel and attached trocar sleeve are prevented by engagement of the cam portion into the barrel recess from distal movement. This is the armed position wherein when force is applied to the protective member the entry tip is allowed to become exposed for use. Pressure on the trocar sleeve as it passes through the patient's abdominal wall urges the barrel proximally until the locking plate disengages from the abutment surface, releasing the actuator rod to move into the first position. The abutment surface may then engage the surface of the barrel adjacent the transverse aperture. When the trocar sleeve has passed through the abdominal wall, the pressure caused by tissue resistance is decreased so that the main spring can cause the shield or protector to extend distally to cover or protect the trocar point. When the trocar point is covered or protected the abutment enters and is engaged by the recess locking the shield or protector to prevent movement relative to the trocar point. This prevents accidental injury to a patient's internal organs.

In preferred embodiments the manual actuator comprises a button mounted in the housing for slidable movement coaxially with the actuator rod. The button may be biassed outwardly of the housing by a spring or other resilient means. A sprung latch member may serve to lock the button in the outward extended position preventing the user from accidentally actuating the button during use.

In a first embodiment the locking member may include a cam portion or other surface arranged to be engaged by the actuation rod as the latter moves into the first position, releasing the latch member and allowing the button to be actuated.

In a second embodiment the locking member comprises a spring biassed towards an expanded position in which a locking position of the spring engages an abutment of the end of the actuator rod.

The end of the actuation rod remote from the button may extend from the housing when in the second position, affording a visual and tactile indication that the device is aimed.

The invention is further described by means of examples but not in any limitative sense with reference to the accompanying drawings of which:

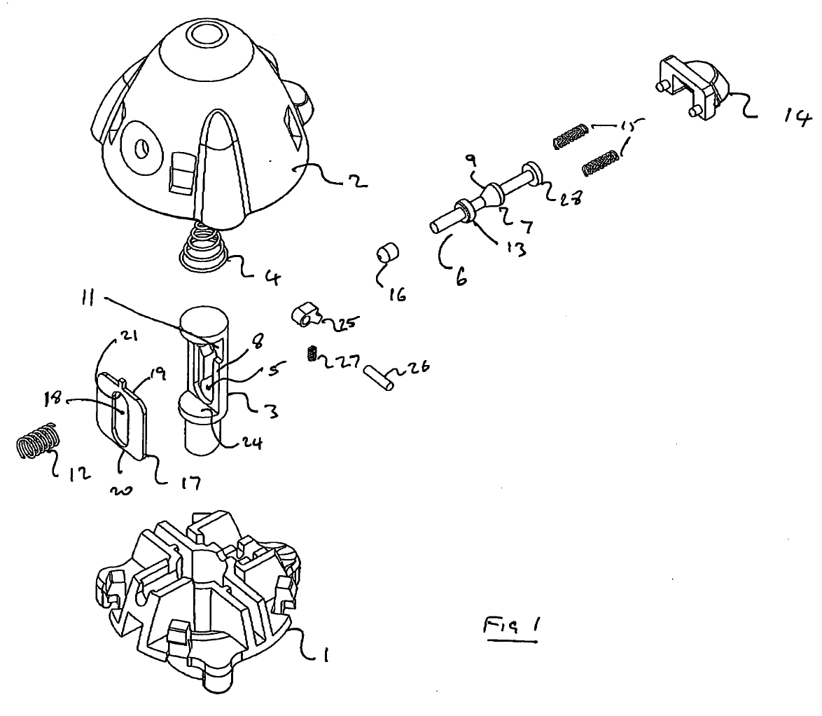

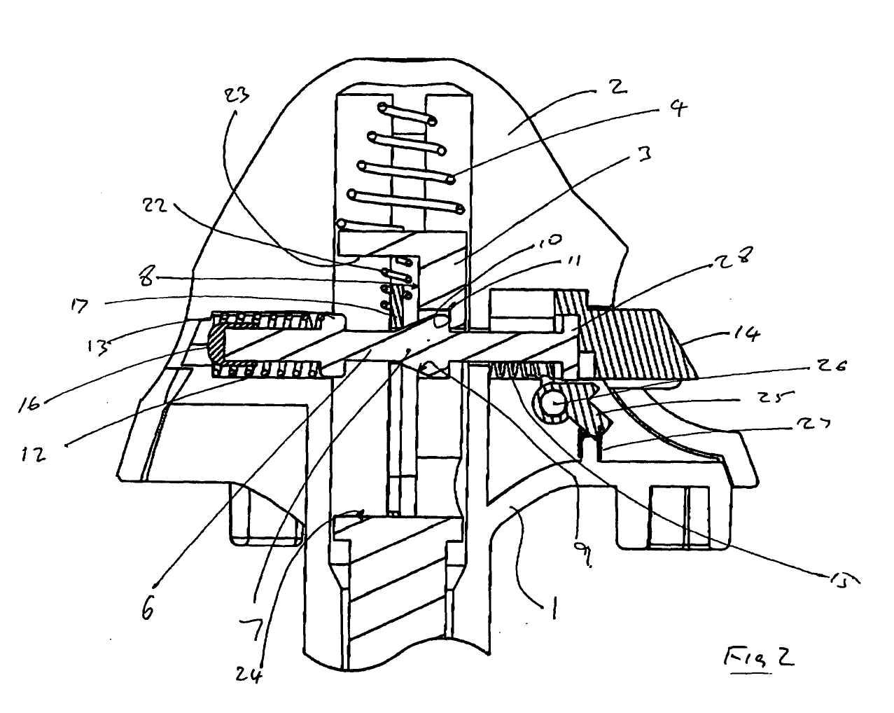

FIG. 1 is an exploded view of a trocar shield assembly in accordance with the invention,

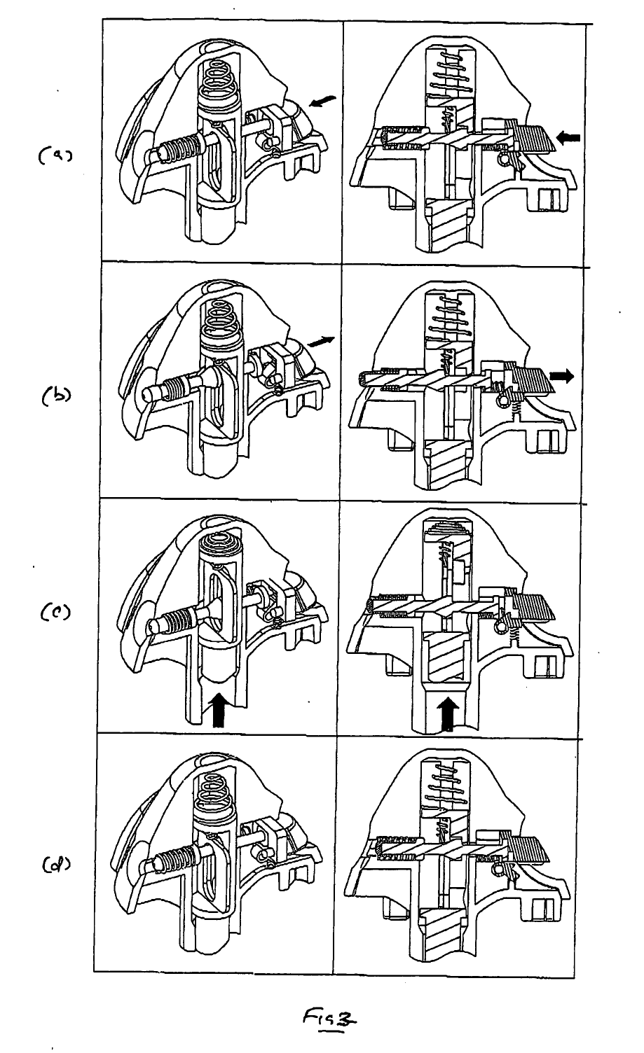

FIG. 2 is a cross-sectional view of the trocar shield shown in FIG. 1,

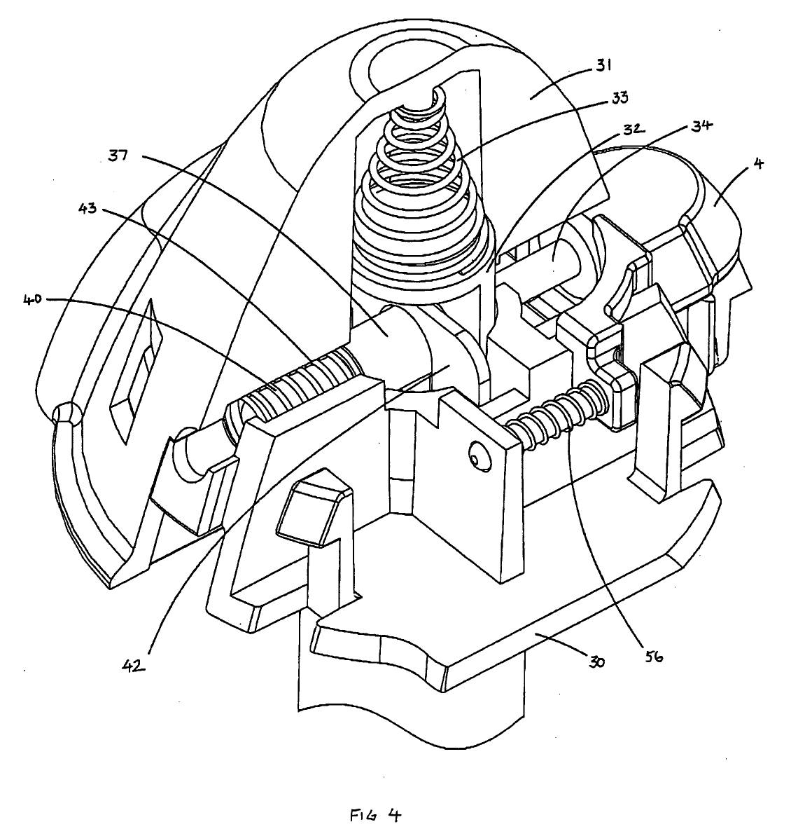

FIG. 3 is a series of four cut-away and sectional views showing use of the assembly,

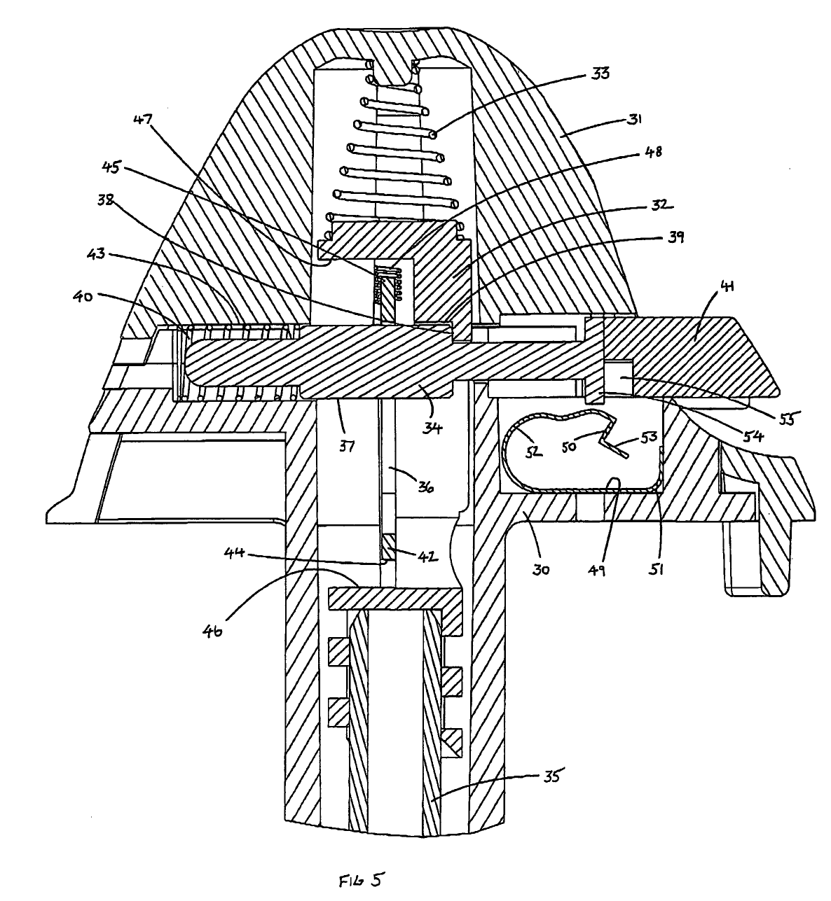

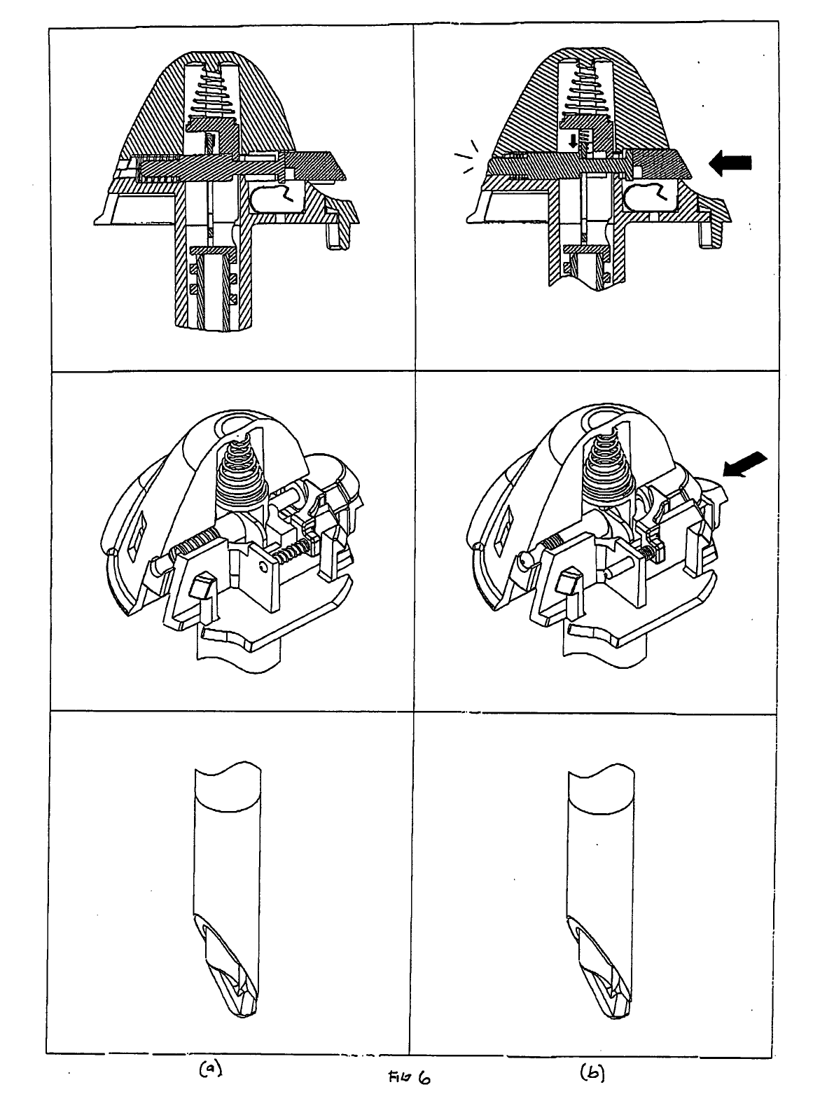

FIG. 4 is a cut away view of a second trocar shield assembly,

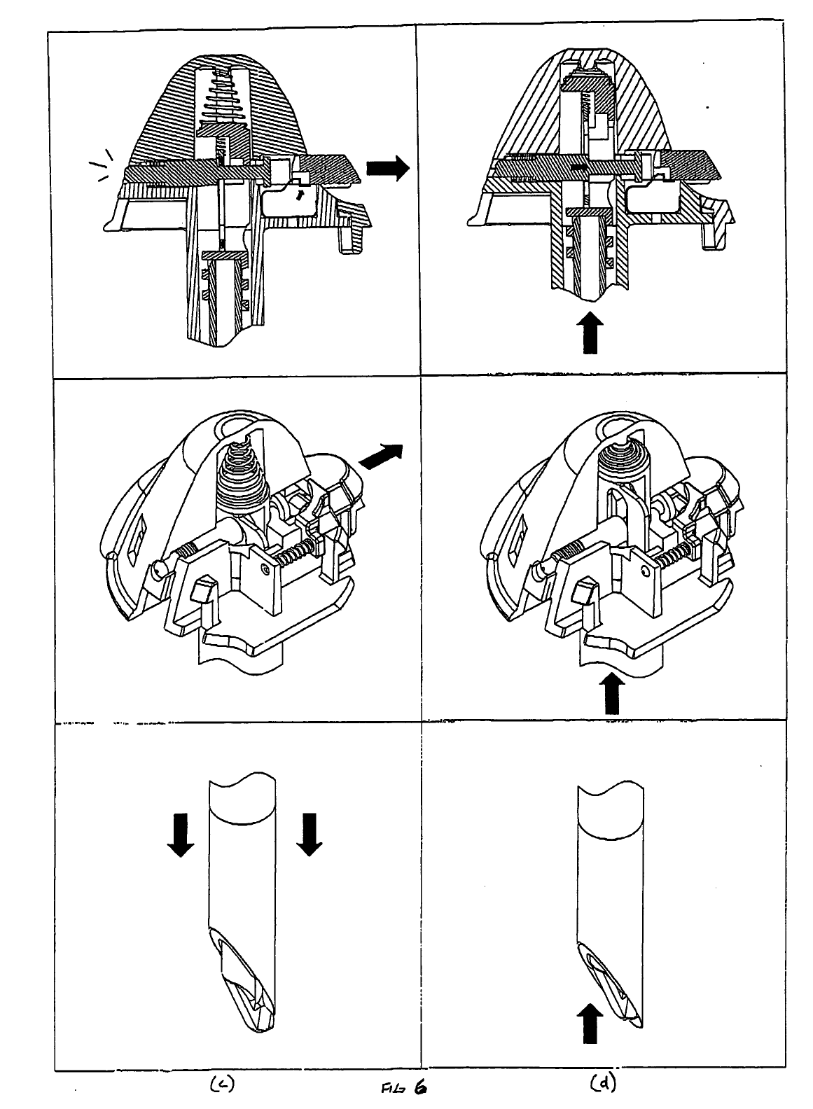

FIG. 5 is a cross-sectional view of the assembly shown in FIG. 4, and

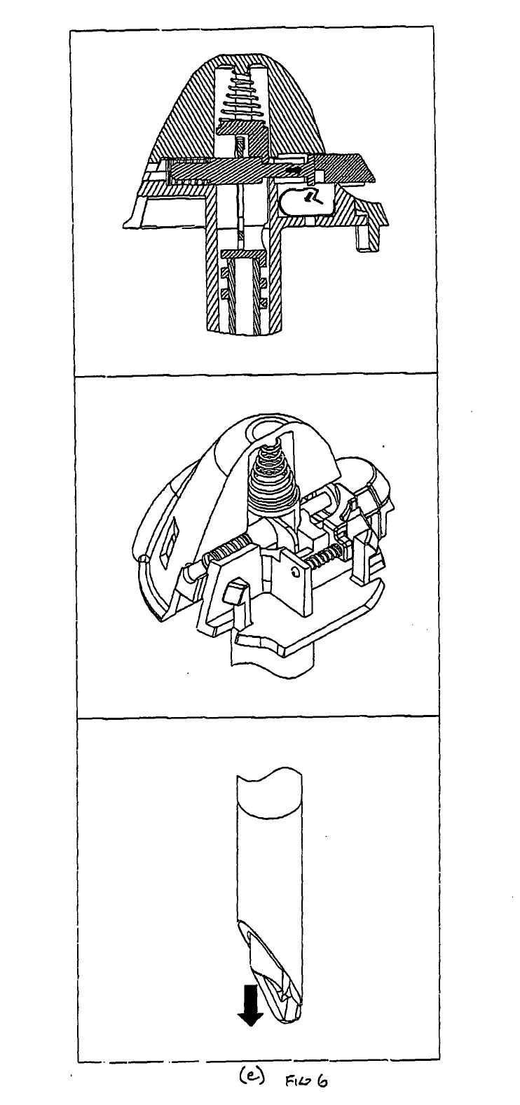

FIG. 6 is a series of five cut-away and sectional views showing use of the assembly shown in FIGS. 3 and 4.

The same reference numerals are used to denote the same components in the drawings.

The assembly shown in FIG. 1 comprises a base moulding (1), an upper housing (2), and an actuation barrel (3) located in an axial bore in the base (1). The barrel (3) may slide axially within the bore and is urged distally by a main spring (4) located between the upper housing and barrel. The base moulding also includes a transverse guideway for the actuation rod (6) which is described in more detail below.

The trocar sleeve comprises a cylindrical body disposed around the moveable axial shaft of the trocar in conventional manner. The shaft is slidable between a distal position in which the trocar point is covered and a proximal position in which the point is exposed for use. The shaft is engaged to the barrel (3) so that both components move together in use. The operation of the trocar tip and sleeve are described below with reference to FIG. 6. A more detailed description of the arrangement may be obtained from U.S. Pat. No. 5,224,951, the disclosure of which is incorporated into this specification by reference. An alternative arrangement is disclosed in U.S. Pat. No. 5,114,407, the disclosure of which is incorporated into this specification by reference.

A releasable locking means is adapted to lock the sleeve in the distal position. The structure of a first preferred mechanism is described below.

The barrel (3) has a transverse aperture or slot (5) through which an actuation rod (6) extends. The actuation rod (6) is movable longitudinally, preferably radially between a first position as shown in FIG. 3a and a second position shown in FIG. 3b.

The actuation rod includes a cam portion (7). In a preferred embodiment the cam portion comprises a generally conical formation (9) extending radially of the rod and increasing in diameter towards the wall (8) of the barrel (3). The wider end of the cam portion (7) forms an abutment surface (10) which preferably extends radially of the actuation rod. The transverse aperture (5) comprises a longitudinal slot so that the barrel may move between proximal and distal positions relative to the actuation rod (6).

A recess in the barrel defines a socket to receive the cam portion (7), preventing axial movement of the barrel. The recess is preferably located at or adjacent the proximal end of the slot (5).

A spring (12) or other resilient means bears against a collar (3) or other formation on the rod (6) to urge the latter towards the first position, as shown in FIG. 3a. A manual actuator, for example, a button (14) is adapted to be depressed in use to urge the rod (6) into the second position, as shown in FIG. 3b. One or more springs (15) urge the button outwardly of the housing. An indicator cap (16) on the end of the rod (6) remote from the button (14) extends from the housing to give a visual display when the rod is in the second position.

A locking plate (17) having a longitudinally extending transverse slot (18) is mounted between end pieces of the barrel for axial or longitudinally movement. The plate (17) is urged distally by a springed or other resilient means (22).

Proximal (19) and distal (20) end surfaces of the plate (17) abut corresponding end surfaces (23, 24) of the end portions of the barrel. The proximal end (21) of slot (18) is urged by the spring (22) into engagement with the shaft of the actuation rod (6), particularly the cam surface (9) Or abutment (10). This forms a latch.

A pawl (25) mounted on a pin (26) is urged into engagement with the actuator button (14) to lock the button outwardly of the housing as shown in FIGS. 3b and c. The end (28) of the rod (6) provides a cam surface to engage the pawl, causing the pawl to rotate unlocking the button (14).

The function of the apparatus is fisher explained with reference to FIG. 3. FIG. 3a shows an isometric cutaway view, together with a cross-section. In the first position the actuation rod (6) is urged by spring (12) into a first position wherein the abutment (10) of the cam portion (7) is urged into and received by the recess (11) of the barrel (3). Axial movement of the barrel and trocar shield in the proximal direction is prevented.

The location of the end (28) of the actuation rod in the first position engages the pawl (25) disengaging the pawl from the actuation member (14).

The locking plate (17) is urged distally by the spring (22) so that the proximal surface (21) of the slot (18) engages the actuation rod.

When the actuation button (14) is depressed as shown by the arrow in FIG. 3a, the actuation rod moves to the left as shown into the second position compressing the spring (12) and exposing the indication cap (16). This will confirm to a user that the device is armed. The locking plate locks behind the abutment (10) of the cam portion to hold the actuation rod in the second position as shown in FIG. 3b. Action of the spring (27) rotates the pawl (25) so that the actuation button (14) cannot be inadvertently depressed. The button is locked in a position isolating it from the rest of the mechanism so that inadvertent contact with the button will not interfere with the resetting of the mechanism.

FIG. 3c shows the upward, proximal force on the central shaft caused by friction between the trocar shield and a patient's body wall as an incision is made. The barrel is moved proximally, compressing the main spring (4) and moving the actuation rod to the distal end of the slots (5) and (18) in the barrel and locking plate respectively.

As the trocar passes through the abdominal wall into the abdominal cavity the force on it reduces and the main spring (4) pushes the barrel forward into the abdominal cavity. This protects the trocar point preventing accidental damage to the internal organs. When the actuation rod reaches the proximal end of the slot (5) in the barrel (3), the abutment of the cam portion (7) enters the recess (11) blocking the actuation barrel.

The end (28) of the actuation rod unlocks the pawl (25) behind the mechanism to be reset for further uses shown in FIG. 3d.

FIGS. 5 and 6 illustrate an alternative embodiment having a generally similar construction to the first embodiment but with a modified actuation rod and manual actuation button assembly.

The trocar shield assembly comprises a base-moulding (30) an upper housing (31) and an actuation barrel (32) located in an axial bore in the base. The barrel (32) is able to slide axially within the bore and is urged distally by a conical main spring (33) located between the upper housing and the barrel.

The cylindrical trocar sleeve (35) is disposed around the trocar and movable between a distal position in which the trocar point is covered and a proximal position in which the point is exposed for use as shown in FIG. 6. The sleeve is integral with the barrel. The barrel has a transverse aperture (43) through which an actuation rod (34) extends. The actuation rod (34) is movable longitudinally, diametrically of the base-moulding and housing as shown in the series of FIG. 6. The actuation rod (34) includes an expanded cylindrical portion (37) defining an abutment surface (38), extending radially of the actuation rod. A recess in the barrel (32) defines a socket (39) to receive the abutment (38) of the cylindrical portion (37) preventing axial movement of the barrel. A spring (40) bears against an abutment formed by the end of the cylindrical portion (37) to urge the actuation rod to the right as shown in FIG. 5 so that the abutment (38) is urged towards the socket (39) in the barrel when the socket and cylindrical member are aligned coaxially. The locking plate (42) aligned longitudinally so that end surfaces (44 and 45) thereof engage end portions (46, 47) of the barrel as in the first embodiment described above. A spring (38) urges the locking plate distally to form a latch.

The arrangement for locking the actuator button (41) outwardly is modified in comparison to the first embodiment described above. A resilient member for example a metal or resilient polymer in spring clip (49) shown in FIG. 5 has a step end piece (50) which is biassed outwardly towards the axis of the actuation rod. The spring (49) has a foot portion (51) received in a recess in a body (30) and an outwardly biassed arm (52) having an abutment for example a stepped formation at the end remote from the foot. The step formation (53) is adapted to engage with the recess (55) between the actuation button or knob (41) and the end (54) of the actuation rod preventing movement of the knob into the body of the trocar handle. The button is then locked in the outwardly extended position. When the latch plate (42) is disengaged from the cylindrical portion (34), spring (40) urges the actuation rod to the right as shown in FIG. 5 depressing the arm (52) of the spring so that the button may be depressed manually to release the sleeve.

The operation of the trocar shield is shown with reference to the series of FIG. 6.

In FIG. 6a the mechanism is in the locked position. The trocar is safe with the protective plunger locked forward. The arming button is disengaged from the toggle spring so that it may be pressed. The central protective plunger of the trocar is locked forward.

In FIG. 6b the arming button is pressed revealing a visual and tactile indicator for the opposite side of the housing warning the user that the trocar is armed. The latching plate moves down under the spring force to lock the locking bar in the armed position. The plunger is free to move rearwardly to expose the trocar blade. In FIG. 6c, the arming button is released, fully arming the trocar. The indicator remains visible. The button toggle spring moves up behind the button preventing inadvertent pressing of the button. The trocar is then ready for use. The plunger is free to move rearwardly exposing the blade.

In FIG. 6d the trocar is pushed into the abdominal cavity of a patient. As the plunger is pressed against the skin it is pushed rearwardly exposing the cutting blade. The conical main spring inside the mechanism is compressed and the locking bar moves off the latching plate to an intermediate position. The mechanism has been tripped.

In FIG. 6e as the plunger enters a free space inside the patient's abdominal cavity, it jumps forward shielding the blade. The locking actuator bar moves back to its original position relocking the trocar. As the actuator bar moves across it displaces the toggle spring from behind the button allowing the button to be pressed and the mechanism reset.

1. A trocar actuator mechanism comprising:
a mounting for a trocar spike;
an axial shaft having a proximal end, a distal end and a bush at the distal end;
the shaft being disposed within a housing and adapted to move relative to the housing between a distal position wherein the trocar spike is protected in use and a proximal position wherein the trocar spike is exposed;
releasable locking means being adapted to lock the shaft when the shaft is moved into the distal position;
an actuator moveable from an armed position wherein the shaft may move within a housing to a disarmed position wherein the shaft is prevented from movement;
wherein the locking means comprises:
an axially movable portion on the proximal end of the shaft;
an actuator extending through a slot in said portion;
wherein the actuator engages proximal and distal ends of the slot to limit movement of the shaft;
the actuator being transversely movable between first and second positions, wherein in the first position the actuator engages the shaft to lock and arm the trocar and wherein manual actuation of the actuator causes the actuator to move to the second position wherein the trocar is disarmed.

2. A trocar actuator mechanism comprising:
a mounting for a trocar spike;
an axial shaft having a proximal end, a distal end and a bush at the distal end;
the shaft being disposed within a housing and adapted to move relative to the housing between a distal position wherein the trocar spike is protected in use and a proximal position wherein the trocar spike is exposed;
releasable locking means being adapted to lock the shaft when the shaft is moved into the distal position;
an actuator moveable from an armed position wherein the shaft may move within a housing to a disarmed position wherein the shaft is prevented from movement;
wherein the locking means comprises:
an axially movable barrel engaging a proximal end of the shaft and urged distally by a main spring,
the barrel having a transverse aperture and further including a socket adjacent, a proximal end of the aperture,
an actuator having an axis and being mounted in the housing and extending transversely through the aperture, the actuator being movable axially between first and second positions,
the actuator having a shaft and a manual actuator at one end thereof, the actuator further including an abutment facing towards the manual actuator,
a locking plate axially movable relative to the barrel, the locking plate including a latch portion adapted to engage the actuation rod preventing movement of the actuator from the second to the first position;
wherein in the first position of the actuator, the actuator engages the socket to lock and arm the shaft, and
wherein actuation of the manual actuator causes the actuator to move to the second disarmed position.

3. A mechanism as claimed in claim 2, wherein the spring or biassing means urges the actuation rod towards the first position wherein the actuator button is caused to extend from the housing.

4. A mechanism as claimed in claims 2 or 3 wherein latch biassing means is adapted to urge the latch into engagement with the actuator.

5. A mechanism as claimed in claim 4, wherein the latch includes longitudinally extending aperture.

6. A mechanism as claimed in claim 5, wherein the transverse aperture comprises a longitudinally extending slot, extending between transverse faces of the barrel.

7. A mechanism as claimed in any of claims 2 to 6 wherein the latch comprises a plate or other slidable member.

8. A mechanism as claimed in claim 7, wherein the latch is located between proximal and distal portions of the barrel.

9. A mechanism as claimed in claim 8, where in the latch is located between proximal and distal end pieces of the barrel.

10. A mechanism as claimed in claim 8 or 9, wherein the latch is permitted to move axially being urged in the distal direction by a spring or other resilient means to cause the plate to engage or be urged towards the actuator.
